(19) United States
(12) Patent Application Publication
(10) Pub. No.: US 2022/0151978 A1
(43) Pub. Date: May 19, 2022

(54) A KIT FOR TREATING PELVIC PAIN ARISING FROM FEMALE REPRODUCTIVE SYSTEM

(71) Applicant: BUZZELET DEVELOPMENT AND TECHNOLOGIES LTD., OR-AKIVA (IL)

(72) Inventors: AHARON M. EYAL, JERUSALEM (IL); NOA RAZ, GIZO (IL); DANA BERNEMAN ZEITOUNI, KFAR YONA (IL); ISO HELLER, NETANYA (IL)

(21) Appl. No.: 17/431,428

(22) PCT Filed: Feb. 17, 2020
(86) PCT No.: PCT/IB2020/051300

§ 371 Date: Aug. 17, 2021

Related U.S. Application Data
(60) Provisional application No. 62/806,841, filed on Feb. 17, 2019.

(51) Int. Cl. A61K 31/352; A61K 31/05; A61K 31/085; A61K 31/045; A61K 31/01; A61K 31/015; A61K 36/29; A61K 36/88; A61K 36/71; A61K 36/8945; A61K 36/65; A61K 36/185; A61K 36/53; A61K 36/232; A61K 36/48; A61K 36/486; A61K 36/28; A61P 15/00; A61P 29/02

(52) U.S. Cl. CPC A61K 31/352; A61P 29/02; A61K 31/085; A61K 31/045; A61K 31/01; A61K 31/015; A61K 36/29; A61K 36/88; A61K 36/71; A61K 36/8945; A61K 36/65; A61K 36/185; A61K 36/53; A61K 36/232; A61K 36/48; A61K 36/486; A61K 36/28; A61P 15/00; A61K 31/05

(57) ABSTRACT

Provided is a kit comprising a first pharmaceutical composition comprising a first cannabinoid and optionally a first terpene and/or a first additive; and a second pharmaceutical composition comprising at least one selected from the group consisting of a second cannabinoid, a second terpene, a second additive and combinations thereof, and uses thereof in the treatment of pelvic pain arising from the female reproductive system. Further provided are methods of treatment of pelvic pain arising from a female reproductive system during a menstrual cycle, the method comprising administering during a first fraction of said menstrual cycle a first pharmaceutical composition comprising a first cannabinoid and optionally a first terpene and/or a first additive, and administering during a second fraction of said menstrual cycle a second pharmaceutical composition comprising at least one selected from the group consisting of a second cannabinoid, a second terpene, a second additive and combinations thereof.

### FIELD OF THE INVENTION

The field of art to which this invention generally pertains is cannabinoids for treatment of pelvic pain, and more particularly to kits and methods comprising cannabinoid compositions for treatment of pelvic pain arising from the female reproductive system.

### BACKGROUND OF THE INVENTION

Pelvic pain is pain in the area of the pelvis. Pelvic pain arising from the female reproductive system is modulated by physiological changes as part of the female menstrual cycle. Dysmenorrhea, also known as painful periods, or menstrual cramps, is a pain during menstruation and is the most common type of pelvic pain. Estimates of the percentage of women of reproductive age affected varying from 20 to 90%. Dysmenorrhea can be classified as either primary or secondary based on the absence or presence of an underlying cause. Primary dysmenorrhea occurs without an associated underlying cause, while secondary dysmenorrhea has a specific underlying cause, typically a condition that affects the uterus or other reproductive organs. The most common cause of secondary dysmenorrhea is endometriosis. Other causes of secondary dysmenorrhea include leiomyoma, adenomyosis, ovarian cysts, and pelvic congestion. Some women also feel pelvic pain during ovulation. Prescribed medication provides relief in only some women.

### SUMMARY OF THE INVENTION

According to an aspect of some embodiments of the present invention, there is provided a kit for treating pelvic pain arising from the female reproductive system, the kit comprising
(a) a first pharmaceutical composition comprising a first cannabinoid and optionally a first terpene and/or a first additive; and
(b) a second pharmaceutical composition comprising at least one selected from the group consisting of a second cannabinoid, a second terpene, a second additive and combinations thereof.

According to a further aspect of some embodiments of the present invention, there is provided a method for treating a a subject with pelvic pain arising from female reproductive system during a menstrual cycle, comprising use of the kit as disclosed herein.

According to a further aspect of some embodiments of the present invention, there is provided a method for treating a subject with pelvic pain arising from female reproductive system during a menstrual cycle, comprising
(i) administering to said subject during a first fraction of said menstrual cycle a first pharmaceutical composition comprising a first cannabinoid and optionally a first terpene and/or a first additive, and
(ii) administering to said subject during a second fraction of said menstrual cycle a second pharmaceutical composition comprising at least one selected from the group consisting of a second cannabinoid, a second terpene, a second additive and combinations thereof.

### DETAILED DESCRIPTION OF THE INVENTION

Unless indicated otherwise, the term “cannabinoid” as used herein refers to a compound that affects the endocannabinoid system. Cannabinoids are agonists or antagonists to receptors in the endocannabinoid system.

Unless indicated otherwise, the term “terpene” as used herein refers to both terpenes and terpenoids.

Unless indicated otherwise, the term “additive” as used herein refers to a non-cannabinoid non-terpene constitute, including herbal preparation, vitamin, food additive, minerals and combinations thereof.

Unless indicated otherwise, the term “dysmenorrhea” as used herein refers to any pain and/or cramp during menstruation, including serious and light pain and/or cramp sensations.

Unless indicated otherwise, the term “endometriosis” as used herein refers to any secondary dysmenorrhea, including leiomyoma, adenomyosis, ovarian cysts, and pelvic congestion.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, “terpene” refers to both terpenes and terpenoids.

As used herein, the term “treating” includes preventing, curing, ameliorating, mitigating, and reducing the instances or severity of a condition or a symptom thereof.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, provided is a kit for treating pelvic pain arising from the female reproductive system, the kit comprising
(a) a first pharmaceutical composition comprising a first cannabinoid and optionally a first terpene and/or a first additive; and
(b) a second pharmaceutical composition comprising at least one selected from the group consisting of a second cannabinoid, a second terpene, a second additive and combinations thereof.

According to an embodiment, said second cannabinoid is present and is different from said first cannabinoid.

According to an embodiment, said second cannabinoid is present and is the same as said first cannabinoid.

According to an embodiment, a dose of said first cannabinoid in said first pharmaceutical composition is at least twice the dose of said second cannabinoid in said second pharmaceutical composition.

According to an embodiment, the kit further comprises (c) a third pharmaceutical composition comprising a second cannabinoid and optionally a third terpene and/or a third additive, wherein a dose of said second cannabinoid in said third pharmaceutical composition is at least twice the dose of said second cannabinoid in said second pharmaceutical composition.

According to an embodiment, the kit further comprises (d) a fourth pharmaceutical composition comprising said second cannabinoid and optionally a fourth terpene and/or a fourth additive.

According to an embodiment, the second cannabinoid in the second composition, when present, the second cannabinoid in the third composition and the second cannabinoid in the fourth composition are each the same cannabinoid.

According to an embodiment, the second cannabinoid in the second composition, when present, the second cannabinoid in the third composition and the second cannabinoid in the fourth composition are different cannabinoids.

According to an embodiment, said second terpene, when present, is the same as said first terpene, when present. According to an embodiment, said second terpene, when present, is different from said first terpene, when present.

According to an embodiment, said fourth terpene, when present, is the same as said second terpene, when present. According to an embodiment, said fourth terpene, when present, is different from said second terpene, when present.

According to an embodiment, said fourth terpene, when present, is the same as said third terpene, when present. According to an embodiment, said fourth terpene, when present, is different from said third terpene, when present.

According to an embodiment, said second additive, when present, is different from said first additive, when present. According to an embodiment, said second additive, when present, is the same as said first additive, when present.

According to an embodiment, said fourth additive, when present, is different from said third additive, when present. According to an embodiment, said fourth additive, when present, is the same as said third additive, when present.

According to an embodiment, at least one of said first cannabinoid and said second cannabinoid, when present, is selected from the group consisting of THC, CBD, CBG, CBC, CBN, CBL, THCV and/or CBDV. According to an embodiment, at least one of said first cannabinoid and said second cannabinoid is selected from the group consisting of THC, CBD and/or CBG.

According to an embodiment, at least one of said first cannabinoid and said second cannabinoid, when present comprises THC.

According to an embodiment, at least one of said second pharmaceutical composition and said fourth pharmaceutical composition comprises CBD. According to some such embodiments, the kit is used for treating endometriosis.

According to an embodiment, at least one of said first terpene, when present, said second terpene, when present, said third terpene, when present, and said fourth terpene, when present, is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, at least one of said first terpene, when present, and said third terpene, when present, is selected from the group consisting of caryophyllene, limonene, myrcene, nerolidol, linalool, terpineol, pinene, humulene, bisabolol, guaiol and combinations thereof.

According to an embodiment, at least one of said second terpene, when present, and said fourth terpene, when present, is selected from the group consisting of linalool, bisabolol, terpineol, myrcene, humulene and combinations thereof.

According to an embodiment, at least one of said first additive, when present, said second additive, when present, said third additive, when present, and said fourth additive, when present, comprises a herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium, Chamaelirium lutea and combinations thereof.

According to an embodiment, at least one of said first terpene and said third terpene is selected from the group consisting of caryophyllene, limonene, myrcene, nerolidol, linalool, terpineol, pinene, humulene, bisabolol, guaiol and combinations thereof. According to an embodiment, at least one of said first terpene and said third terpene is selected from the group consisting of caryophyllene, limonene, myrcene, nerolidol and combinations thereof.

According to an embodiment, at least one of said second terpene and said fourth terpene is selected from the group consisting of linalool, bisabolol, terpineol, myrcene, humulene and combinations thereof. According to an embodiment, at least one of said second terpene and said fourth terpene is selected from the group consisting of linalool, bisabolol, terpineol and combinations thereof.

According to an embodiment, at least one of said first additive, when present, said second additive, when present, said third additive, when present, and said fourth additive, when present, comprises herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium, Chamaelirium lutea and combinations thereof.

According to an embodiment, at least one of said first additive, when present, said second additive, when present, said third additive, when present, and said fourth additive, when present comprises a Vitex agnus-castus and/or Chamaelirium lutea preparation.

According to an embodiment, at least one of said second additive, when present and said fourth additive, when present, comprises a Vitex agnus-castus and/or Chamaelirium lutea preparation.

According to an embodiment, at least one of said first additive, when present and said third additive, when present, comprises herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium Chamaelirium lutea and combinations thereof. According to an embodiment, at least one of said first additive and said third additive comprises herbal preparation selected from the group consisting of extracts of Caulophyllum thalictroides, Cimicifuga (Actaea) racemosa, Paeonia lactiflora, Viburnum opulus, Viburnum prunifolium and combinations thereof.

According to an embodiment, at least one of said first pharmaceutical composition, said second pharmaceutical composition, said third pharmaceutical composition and said fourth pharmaceutical composition, when present, is provided in a form selected from the group consisting of a topical, a cream, a patch, a vaginal applicator, a tablet, a gel capsule, a sublingual oil, a spray, a tincture, a varnish, an edible, a beverage, a cigarette, a vaporizer liquid, a nasal preparation or combinations thereof.

According to an embodiment, said first pharmaceutical composition is provided in the same form as said third pharmaceutical composition. According to an embodiment, said second pharmaceutical composition is provided in the same form as said fourth pharmaceutical composition. According to an embodiment, said first pharmaceutical composition is provide in a different form from said third pharmaceutical composition. According to an embodiment, said first pharmaceutical composition and/or said third pharmaceutical composition comprises at least two, at least three, or at least four forms selected from the group consisting of a topical, a cream, a patch, a vaginal applicator, a tablet, a gel capsule, a sublingual oil, and a spray, a tincture, a varnish, an edible, a beverage, a cigarette, a vaporizer liquid, and a nasal preparation. According to an embodiment, said second pharmaceutical composition and/or said fourth pharmaceutical composition comprises at least two, at least three, or at least four forms selected from the group consisting of a topical, a cream, a patch, a vaginal applicator, a tablet, a gel capsule, a sublingual oil, a spray, a tincture, a varnish, an edible, a beverage, a cigarette, a vaporizer liquid, a nasal preparation.

According to an embodiment, at least one of said first pharmaceutical composition and said third pharmaceutical composition comprise a form selected from the group consisting of a topical, a cream, a patch, a tablet, a gel capsule, a sublingual oil, a spray, a tincture, a varnish, an edible, a beverage, a cigarette, a vaporizer liquid, a nasal preparation, or combinations thereof.

According to an embodiment, at least one of said first pharmaceutical composition, said second pharmaceutical composition, said third pharmaceutical composition and said fourth pharmaceutical composition is provided in a form selected from the group consisting of a topical, a cream, a patch, a tablet, a gel capsule, a sublingual oil, a spray, a tincture, or combinations thereof.

According to an embodiment, the kit is for monthly use.

According to an embodiment, at least one of said first pharmaceutical composition and said third pharmaceutical composition comprises between 1 milligram and 100 milligrams of said cannabinoid.

According to an embodiment, at least one of said first pharmaceutical composition, said second pharmaceutical composition, said third pharmaceutical composition and said fourth pharmaceutical composition comprises between 1 milligram and 100 milligrams of said cannabinoid.

According to an embodiment, said first pharmaceutical composition and/or said third pharmaceutical composition comprises at least 1 milligram, at least 2, at least, 3, at least 5, at least 10, at least 15, at least 20, at least 25, at least 30, at least 35, at least 40, at least 50, or at least 6o milligrams of said cannabinoid. According to an embodiment, said first pharmaceutical composition and/or said third pharmaceutical composition comprises less than 100 milligrams, less than 90, less than 80, less than 70, less than 60, less than 50, less than 40, less than 30, less than 20, or less than 10 milligrams of said cannabinoid.

According to an embodiment, at least one of said first pharmaceutical composition, said second pharmaceutical composition, said third pharmaceutical composition and said fourth pharmaceutical composition comprises at least 1 milligram, at least 2, at least, 3, at least 5, at least 10, at least 15, at least 20, at least 25, at least 30, at least 35, at least 40, at least 50, or at least 6o milligrams of said cannabinoid. According to an embodiment, at least one of said first pharmaceutical composition, said second pharmaceutical composition, said third pharmaceutical composition and said forth pharmaceutical composition comprises less than 100 milligrams, less than 90, less than 80, less than 70, less than 60, less than 50, less than 40, less than 30, less than 20, or less than 10 milligrams of said cannabinoid.

According to an embodiment, at least one of said first pharmaceutical composition, said second pharmaceutical composition, said third pharmaceutical composition and said fourth pharmaceutical composition comprises between 0.1 milligram and 10 milligrams of said terpene. According to an embodiment, at least one of said first pharmaceutical composition, said second pharmaceutical composition, said third pharmaceutical composition and said fourth pharmaceutical composition comprises at least 0.1 milligram said terpene, at least 0.2, at least 0.5, at least 1, at least 1.5, at least 2, at least 3, at least 4, at least 5, at least 6, at least 7, at least 8, at least 9 milligrams. According to an embodiment, at least one of said first pharmaceutical composition, said second pharmaceutical composition, said third pharmaceutical composition and said fourth pharmaceutical composition comprises less than 10 milligrams said terpene, less than 9, less than 8, less than 7, less than 6, less than s, less than 4, less than 3, less than 2, or less than 1 milligram.

According to an embodiment, at least one of said first terpene, said second terpene, said third terpene and said fourth terpene comprises between 0.1 milligram and 10 milligrams. According to an embodiment, at least one of said first terpene, said second terpene, said third terpene and said fourth terpene comprises at least 0.1 milligram, at least 0.2, at least 0.5, at least 1, at least 1.5, at least 2, at least 3, at least 4, at least 5, at least 6, at least 7, at least 8, at least 9 milligrams. According to an embodiment, at least one of said first terpene, said second terpene, said third terpene and said fourth terpene comprises less than 10 milligrams, less than 9, less than 8, less than 7, less than 6, less than 5, less than 4, less than 3, less than 2, or less than 1 milligram.

According to an embodiment, at least one of said first pharmaceutical composition, said second pharmaceutical composition, said third pharmaceutical composition, and said fourth pharmaceutical composition comprises between 0.1 milligram and 10 milligrams of said terpene.

According to an embodiment, at least one of said first pharmaceutical composition, said second pharmaceutical composition, said third pharmaceutical composition, and said fourth pharmaceutical composition comprises between 1 milligram and 1000 milligrams of said additive.

According to an embodiment, at least one of said first pharmaceutical composition, said second pharmaceutical composition, said third pharmaceutical composition and said fourth pharmaceutical composition comprises at least 1 milligram of said additive, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 milligrams of said additive. According to an embodiment, at least one of said first pharmaceutical composition, said second pharmaceutical composition, said third pharmaceutical composition and said fourth pharmaceutical composition comprises less than 1000 milligrams of said additive, less than 900, less than 800, less than 700, less than 600, less than 500, less than 400, less than 300, less than 200, or less than 100, less than 50, less than 25, less than 10, or less than 5 milligram of said additive.

According to an embodiment, at least one of said first additive, said second additive, said third additive and said fourth additive comprises between 1 milligram and 1000 milligrams. According to an embodiment, at least one of said first additive, said second additive, said third additive and said fourth additive comprises at least 1 milligram, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 milligrams. According to an embodiment, at least one of said first additive, said second additive, said third additive and said fourth additive comprises less than 1000 milligrams of said additive, less than 900, less than 800, less than 700, less than 600, less than 500, less than 400, less than 300, less than 200, or less than mo, less than 50, less than 25, less than 10 or less than 5 milligram.

According to an embodiment, at least one of said first pharmaceutical composition, said second pharmaceutical composition, said third pharmaceutical composition and said fourth pharmaceutical composition comprises two or more said cannabinoids. According to some such embodiments, a dose of said cannabinoids is a total dose of each of said cannabinoids.

According to an embodiment, at least one of said first pharmaceutical composition, said second pharmaceutical composition, said third pharmaceutical composition and said fourth pharmaceutical composition comprises two or more said terpenes. According to some such embodiments, a dose of said terpenes is a total dose of each of said terpenes.

According to an embodiment, at least one of said first pharmaceutical composition, said second pharmaceutical composition, said third pharmaceutical composition and said fourth pharmaceutical composition comprises two or more said additives. According to some such embodiments, a dose of said additives is a total dose of each of said additives.

According to an embodiment, said kit is for use in treating dysmenorrhea. According to some such embodiments, the kit comprises said first pharmaceutical composition and said second pharmaceutical composition. According to an embodiment, said kit is for use in treating painful periods, and/or treating menstrual cramps.

According to an embodiment, said kit is for use in treating endometriosis. According to some such embodiments, the kit comprises said first pharmaceutical composition, said second pharmaceutical composition, and said third pharmaceutical composition. According to some such embodiments, the kit comprises said first pharmaceutical composition, said second pharmaceutical composition, said third pharmaceutical composition and said fourth pharmaceutical composition. According to an embodiment, said kit is for use in treating at least one condition selected from the group consisting of uterine fibroids, adenomyosis, leiomyoma, ovarian cysts, and pelvic congestion.

According to an aspect of some embodiments of the present invention, provided is a method for treating a subject with pelvic pain arising from female reproductive system during a menstrual cycle, comprising use of the kit as disclosed herein. According to an embodiment, said pelvic pain comprises dysmenorrhea. According to an embodiment, said pelvic pain arises from endometriosis. According to an embodiment, said pelvic pain comprises painful periods and/or menstrual cramps.

According to an embodiment, provided is a method for treating a subject with pelvic pain arising from female reproductive system during a menstrual cycle, comprising

(i) administering to said subject during a first fraction of said menstrual cycle a first pharmaceutical composition comprising a first cannabinoid and optionally a first terpene and/or a first additive, and

(ii) administering to said subject during a second fraction of said menstrual cycle a second pharmaceutical composition comprising at least one selected from the group consisting of a second cannabinoid, a second terpene, a second additive and combinations thereof.

According to an embodiment, said second cannabinoid is present and is different from said first cannabinoid.

According to an embodiment, said second cannabinoid is present and is the same as said first cannabinoid.

According to an embodiment, a dose of said first cannabinoid in said first pharmaceutical composition is at least twice the dose of said second cannabinoid in said second pharmaceutical composition.

According to an embodiment, the method further comprises administering to said subject during a third fraction of said menstrual cycle a third pharmaceutical composition comprising a second cannabinoid and optionally a third terpene and/or a third additive, wherein a dose of said second cannabinoid in said third pharmaceutical composition is at least twice the dose of said second cannabinoid in said second pharmaceutical composition.

According to an embodiment, said method further comprises administering to said subject during a fourth fraction of said menstrual cycle a fourth pharmaceutical composition comprising said second cannabinoid and optionally a fourth terpene and/or a fourth additive.

According to an embodiment, said pelvic pain comprises dysmenorrhea.

According to an embodiment, said first fraction of the menstrual cycle comprise at least one day of the menstruation phase and optionally at least one of the days immediately before or immediately after menstruation.

According to an embodiment, said second fraction of the menstrual cycle comprises at least one day of the follicular phase and/or at least one day of the luteal phase.

According to an embodiment, said pelvic pain arises from endometriosis.

According to an embodiment, said pelvic pain arises from a condition selected from the group consisting of uterine fibroids, adenomyosis, leiomyoma, ovarian cysts, and/or pelvic congestion.

According to an embodiment, first fraction of said menstrual cycle comprises at least one day of the menstruation period.

According to an embodiment, said third fraction of said menstrual cycle comprises at least one day of the ovulation phase.

According to an embodiment, said second fraction of said menstrual cycle comprises at least one day of the follicular phase.

According to an embodiment, said fourth fraction of said menstrual cycle comprises at least one day of the luteal phase.

According to an embodiment, said second cannabinoid, when present is the same as said first cannabinoid. According to an embodiment, said second cannabinoid, when present is different from said first cannabinoid.

According to an embodiment, said fourth terpene, when present, is the same as said second terpene, when present. According to an embodiment, said fourth terpene, when present, is different from said second terpene, when present.

According to an embodiment, said second terpene, when present, is different from said first terpene, when present. According to an embodiment, said second terpene, when present, is the same as said first terpene, when present.

According to an embodiment, said second additive, when present is different from said first additive, when present. According to an embodiment, said second additive, when present is the same as said first additive, when present.

According to an embodiment, said fourth terpene, when present, is different from said third terpene, when present. According to an embodiment, said fourth terpene, when present, is the same as said third terpene, when present.

According to an embodiment, said fourth additive, when present, is different from said third additive, when present. According to an embodiment, said fourth additive, when present, is the same as said third additive, when present.

According to an embodiment, at least one of said first cannabinoid and said second cannabinoid, when present, is selected from the group consisting of THC, CBD, CBG, CBC, CBN, CBL, THCV and/or CBDV. According to an embodiment, at least one of said first cannabinoid and said second cannabinoid is selected from the group consisting of THC, CBD and/or CBG.

According to an embodiment, at least one of said first cannabinoid and said second cannabinoid, when present, comprises THC.

According to an embodiment, at least one of said second pharmaceutical composition and said fourth pharmaceutical composition comprises CBD.

According to an embodiment, at least one of said first terpene, when present, said second terpene, when present, said third terpene, when present, and said fourth terpene, when present is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, at least one of said first terpene, when present and said third terpene, when present, is selected from the group consisting of caryophyllene, limonene, myrcene, nerolidol, linalool, terpineol, pinene, humulene, bisabolol, guaiol and combinations thereof. According to an embodiment, at least one of said first terpene, when present and said third terpene, when present, is selected from the group consisting of caryophyllene, limonene, myrcene, nerolidol and combinations thereof.

According to an embodiment, at least one of said second terpene, when present, and said fourth terpene, when present, is selected from the group consisting of linalool, bisabolol, terpineol, myrcene humulene and combinations thereof. According to an embodiment, at least one of said second terpene, when present, and said fourth terpene, when present, is selected from the group consisting of linalool, bisabolol, terpineol and combinations thereof.

According to an embodiment, at least one of said first additive, when present, said second additive, when present, said third additive, when present, and said fourth additive, when present, comprises a herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium, Chamaelirium lutea and combinations thereof.

According to an embodiment, at least one of said first additive, when present, said second additive, when present, said third additive, when present, and said fourth additive, when present, comprises a herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium, Chamaelirium lutea and combinations thereof.

According to an embodiment, at least one of said first additive, when present, said second additive, when present, said third additive, when present, and said fourth said additive, when present comprises a Vitex agnus-castus and/or Chamaelirium lutea preparation.

According to an embodiment, at least one of said second additive, when present, and said fourth additive, when present, comprises a Vitex agnus-castus and/or Chamaelirium lutea preparation.

According to an embodiment, at least one of said first additive and said third additive comprises herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium, and/or Chamaelirium and combinations thereof. According to an embodiment, at least one of said first additive and said third additive comprises herbal preparation selected from the group consisting of extracts of Caulophyllum thalictroides, Cimicifuga (Actaea) racemosa, Paeonia lactiflora, Viburnum opulus, Viburnum prunifolium and combinations thereof.

According to an embodiment, at least one of said first composition, said second composition, said third composition and said fourth composition is administered in a form selected from the group consisting of a topical, a cream, a patch, a vaginal applicator, a tablet, a gel capsule, a sublingual oil, a spray, a tincture, a varnish, an edible, a beverage, a cigarette, a vaporizer liquid, a nasal preparation or combinations thereof.

According to an embodiment, at least one of said first pharmaceutical composition and said third pharmaceutical composition is administered in a form selected from the group consisting of a topical, a cream, a patch, a tablet, a gel capsule, a sublingual oil, a spray or combinations thereof.

According to an embodiment, said first pharmaceutical composition is administered in the same form as said third pharmaceutical composition. According to an embodiment, said second pharmaceutical composition is administered in the same form as said fourth pharmaceutical composition. According to an embodiment, said first pharmaceutical composition is administered in a different form from said third pharmaceutical composition. According to an embodiment, said first pharmaceutical composition and/or said third pharmaceutical composition comprises at least two, at least three, or at least four forms selected from the group consisting of a topical, a cream, a patch, a vaginal applicator, a tablet, a gel capsule, a sublingual oil, and a spray, a tincture, a varnish, an edible, a beverage, a cigarette, a vaporizer liquid, and a nasal preparation. According to an embodiment, said second pharmaceutical composition is administered in at least two, at least three, or at least four forms selected from the group consisting of a topical, a cream, a patch, a vaginal applicator, a tablet, a gel capsule, a sublingual oil, and a spray, a tincture, a varnish, an edible, a beverage, a cigarette, a vaporizer liquid and a nasal preparation.

According to an embodiment, at least one of said first composition and said third composition is administered in a form selected from the group consisting of a topical, a cream, a patch, a tablet, a gel capsule, a sublingual oil, a spray, a tincture, a varnish, an edible, a beverage, a cigarette, a vaporizer liquid, a nasal preparation or combinations thereof.

According to an embodiment, at least one of said first pharmaceutical composition and said third pharmaceutical composition comprises between 1 milligram and 100 milligrams of said cannabinoid. According to an embodiment, said first pharmaceutical composition and/or said third pharmaceutical composition comprises at least 1 milligram per day, at least 2, at least, 3, at least 5, at least 10, at least 15, at least 20, at least 25, at least 30, at least 35, at least 40, at least 50, or at least 6o milligrams of said cannabinoid. According to an embodiment, said first pharmaceutical composition and/or said third pharmaceutical composition comprises less than 100 milligrams, less than 90, less than 80, less than 70, less than 60, less than 50, less than 40, less than 30, less than 20, or less than 10 milligrams of said cannabinoid. According to an embodiment, said first pharmaceutical composition and said pharmaceutical composition are for administration once per day.

According to an embodiment, at least one of said first pharmaceutical composition, said second pharmaceutical composition, said third pharmaceutical composition and said fourth pharmaceutical composition comprises between 1 milligram and 100 milligrams of said cannabinoid. According to an embodiment, at least one of said first pharmaceutical composition, said second pharmaceutical composition, said third pharmaceutical composition and said fourth pharmaceutical composition comprises at least 1 milligram, at least 2, at least, 3, at least 5, at least 10, at least 15, at least 20, at least 25, at least 30, at least 35, at least 40, at least 50, or at least 6o milligrams of said cannabinoid. According to an embodiment, at least one of said first pharmaceutical composition, said second pharmaceutical composition, said third pharmaceutical composition and said fourth pharmaceutical composition comprises less than 100 milligrams, less than 90, less than 80, less than 70, less than 60, less than 50, less than 40, less than 30, less than 20, or less than 10 milligrams of said cannabinoid. According to an embodiment, said first pharmaceutical composition, said second pharmaceutical composition, said third pharmaceutical composition and said fourth pharmaceutical composition are for administration once per day.

According to an embodiment, at least one of said first pharmaceutical composition, said second pharmaceutical composition, said third pharmaceutical composition and said fourth pharmaceutical composition comprises between 0.1 milligram and 10 milligrams of said terpene. According to an embodiment, at least one of said first pharmaceutical composition, said second pharmaceutical composition, said third pharmaceutical composition and said forth pharmaceutical composition comprises at least 0.1 milligram said terpene, at least 0.2, at least 0.5, at least 1, at least 1.5, at least 2, at least 3, at least 4, at least 5, at least 6, at least 7, at least 8, at least 9 milligrams. According to an embodiment, at least one of said first pharmaceutical composition, said second pharmaceutical composition, said third pharmaceutical composition and said forth pharmaceutical composition comprises less than 10 milligrams said terpene, less than 9, less than 8, less than 7, less than 6, less than 5, less than 4, less than 3, less than 2, or less than 1 milligram. According to an embodiment, said first pharmaceutical composition, said second pharmaceutical composition, said third pharmaceutical composition and said fourth pharmaceutical composition are for administration once per day.

According to an embodiment, at least one of said first terpene, said second terpene, said third terpene and said fourth terpene comprises between 0.1 milligram and 10 milligrams. According to an embodiment, at least one of said first terpene, said second terpene, said third terpene and said fourth terpene comprises at least 0.1 milligram, at least 0.2, at least 0.5, at least 1, at least 1.5, at least 2, at least 3, at least 4, at least 5, at least 6, at least 7, at least 8, at least 9 milligrams. According to an embodiment, at least one of said first terpene, said second terpene, said third terpene and said fourth terpene comprises less than 10 milligrams, less than 9, less than 8, less than 7, less than 6, less than 5, less than 4, less than 3, less than 2, or less than 1 milligram. According to an embodiment, said first pharmaceutical composition, said second pharmaceutical composition, said third pharmaceutical composition and said fourth pharmaceutical composition are for administration once per day.

According to an embodiment, at least one of said first pharmaceutical composition, said second pharmaceutical composition, said third pharmaceutical composition and said fourth pharmaceutical composition comprises between 1 milligram and 1000 milligrams of said additive. According to an embodiment, at least one of said first pharmaceutical composition, said second pharmaceutical composition, said third pharmaceutical composition and said forth pharmaceutical composition comprises at least 1 milligram of said additive, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 milligrams of said additive. According to an embodiment, at least one of said first pharmaceutical composition, said second pharmaceutical composition, said third pharmaceutical composition and said forth pharmaceutical composition comprises less than 1000 milligrams of said additive, less than 900, less than 800, less than 700, less than 600, less than 500, less than 400, less than 300, less than 200, or less than 100, less than 50, less than 25, less than 10, or less than 5 milligram of said additive. According to an embodiment, said first pharmaceutical composition, said second pharmaceutical composition, said third pharmaceutical composition and said fourth pharmaceutical composition are for administration once per day.

According to an embodiment, at least one of said first additive, said second additive, said third additive and said fourth additive comprises between 1 milligram and 1000 milligrams. According to an embodiment, at least one of said first additive, said second additive, said third additive and said fourth additive comprises at least 1 milligram, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 milligrams. According to an embodiment, at least one of said first additive, said second additive, said third additive and said fourth additive comprises less than 1000 milligrams of said additive, less than 900, less than 800, less than 700, less than 600, less than 500, less than 400, less than 300, less than 200, or less than 100, less than 50, less than 25, less than 10 or less than 5 milligram. According to an embodiment, said first pharmaceutical composition, said second pharmaceutical composition, said third pharmaceutical composition and said fourth pharmaceutical composition are for administration once per day.

### EXAMPLES 1-12

Tables 1-6 present examples of the compositions of cannabinoid comprising kits, as described herein.

### Example 1: Kit #1

| | Pharmaceutical | Dosage | Cannabinoid | | |
|---|---|---|---|---|---|
| Kit | compositions | form | [mg per day] | Terpene | Additive |
| 1 | First | Tablet | THC | Caryophyl- | Cimicifuga |
| | | | [15 mg] | lene | (Actaea) |
| | | | | | racemosa |
| | Second | Cream | | Linalool | Vitex |
| | | | | | agnus-castus |
| | Third | Tablet | CBD | Myrcene | |
| | | | [10 mg] | | |
| | Fourth | Cream | | Linalool | Vitex |
| | | | | | agnus-castus |

### Example 2: Kit #2

| | Pharmaceutical | Dosage | Cannabinoid | | |
|---|---|---|---|---|---|
| Kit | compositions | form | [mg per day] | Terpene | Additive |
| 2 | First | Gel | CBD | Limonene | Paeonia |
| | | capsule | [20 mg] | | lactiflora, |
| | Second | Gel | CBD | Bisabolol | Chamaelirium |
| | | capsule | [5 mg] | | lutea |

### Example 3: Kit #3

| | Pharmaceutical | Dosage | Cannabinoid | | |
|---|---|---|---|---|---|
| Kit | compositions | form | [mg per day] | Terpene | Additive |
| 3 | First | Vaginal | THC | Myrcene | |
| | | applicator | [20 mg] | | |
| | Second | Cream | | Bisabolol | Vitex |
| | | | | | agnus-castus |
| | Third | Vaginal | CBD | Limonene | Angelica |
| | | applicator | [5 mg] | | sinensis |
| | Fourth | Cream | CBD | Bisabolol | Vitex |
| | | | [2 mg] | | agnus-castus |

### Example 4: Kit #4

| | Pharmaceutical | Dosage | Cannabinoid | | |
|---|---|---|---|---|---|
| Kit | compositions | form | [mg per day] | Terpene | Additive |
| 4 | First | Patch | CBD | Nerolidol | Viburnum |
| | | | [25 mg] | | opulus |
| | Second | Patch | CBD | Terpineol | Vitex |
| | | | [10 mg] | | agnus-castus |

### Example 5: Kit #5

| | Pharmaceutical | Dosage | Cannabinoid | | |
|---|---|---|---|---|---|
| Kit | compositions | form | [mg per day] | Terpene | Additive |
| 3 | First | Vaginal | THC | Myrcene | |
| | | applicator | [20 mg] | and pinene | |
| | Second | Sublingual | CBD | | Chamaelir- |
| | | oil | [2 mg] | | ium lutea |
| | Third | Vaginal | CBD | Myrcene | Angelica |
| | | applicator | [10 mg] | and | sinensis |
| | | | | Limonene | |
| | Fourth | Sublingual | CBD | | Chamaelir- |
| | | oil | [2 mg] | | ium lutea |

### Example 6: Kit #6

| | Pharmaceutical | Dosage | Cannabinoid | | |
|---|---|---|---|---|---|
| Kit | compositions | form | [mg per day] | Terpene | Additive |
| 4 | First | Tablet | CBD | Caryophyl- | Paeonia |
| | | | [20 mg] | lene | lactiflora |
| | Second | Tablet | CBD | Terpineol | Vitex |
| | | | [5 mg] | | agnus-castus |

### Example 7: Treating a Woman with Endometriosis

A woman at the age of 25 diagnosed with endometriosis is treated by administering four pharmaceutical compositions within a kit. The first pharmaceutical composition contains 15 mg THC, a selected amount of Myrcene and a selected amount of Angelica sinensis, and is administrated during the menstruation period; The second pharmaceutical composition contains Terpineol and a selected amount of Chamaelirium lutea, and is administrated during the follicular phase; The third pharmaceutical composition contains 10 mg CBD and a selected amount of Myrcene and is administrated during the ovulation phase; The forth pharmaceutical composition contains Bisabolol and a selected amount of Chamaelirium lutea, and is administrated during the luteal phase.

### Example 8: Treating a Woman with Dysmenorrhea

A woman at the age of 18 diagnosed with dysmenorrhea is treated by administering two pharmaceutical compositions within a kit. The first pharmaceutical composition contains 20 mg CBD, a selected amount of Nerolidol and a selected amount of Piscidia erythrina, and is administrated during the menstruation phase and menstruation-phase surrounding days; The second pharmaceutical composition contains 5 mg CBD, a selected amount of Linalool and a selected amount of Vitex agnus-castus, and is administrated during the follicular phase.

### Example 9: Treating a Woman with Endometriosis

A woman at the age of 3o diagnosed with endometriosis is treated by administering four pharmaceutical compositions within a kit. The first pharmaceutical composition contains 20 mg THC, a selected amount of Humulene and a selected amount of Viburnum opulus, and is administrated during the menstruation period; The second pharmaceutical composition contains 5 mg THC, a selected amount of Myrcene and a selected amount of Chamaelirium lutea, and is administrated during the follicular phase; The third pharmaceutical composition contains 15 mg CBD and a selected amount of Pinene and a selected amount of Viburnum opulus, and is administrated during the ovulation phase; The forth pharmaceutical composition contains 5 mg CBD, a selected amount of Myrcene and a selected amount of Chamaelirium lutea and is administrated during the luteal phase.

### Example 10: Treating a Woman with Dysmenorrhea

An adolescent at the age of 16 diagnosed with dysmenorrhea is treated by administering two pharmaceutical compositions within a kit. The first pharmaceutical composition contains 20 mg CBD, a selected amount of Myrcene and Pinene and a selected amount of Paeonia lactiflora, and is administrated during the menstruation phase and menstruation-phase surrounding days; The second pharmaceutical composition contains 5 mg CBD, a selected amount of Bisabolol and a selected amount of Vitex agnus-castus, and is administrated during the follicular phase.

### Example 11: Treating a Woman with Endometriosis

A woman at the age of 32 diagnosed with endometriosis is treated by administering four pharmaceutical compositions within a kit. The first pharmaceutical composition contains 15 mg THC, a selected amount of Limonene and a selected amount of Piscidia erythrina, and is administrated during the menstruation period; The second pharmaceutical composition contains a selected amount of Myrcene and a selected amount of Chamaelirium lutea, and is administrated during the follicular phase; The third pharmaceutical composition contains 15 mg CBD and a selected amount of Limonene and a selected amount of Viburnum opulus, and is administrated during the ovulation phase; The forth pharmaceutical composition contains a selected amount of Myrcene and a selected amount of Chamaelirium lutea and is administrated during the luteal phase.

### Example 12: Treating a Woman with Dysmenorrhea

An women at the age of 22 diagnosed with dysmenorrhea is treated by administering two pharmaceutical compositions within a kit. The first pharmaceutical composition contains 20 mg CBD and a selected amount of Pinene, and is administrated during the menstruation phase and menstruation-phase surrounding days; The second pharmaceutical composition contains a selected amount of Linalool and a selected amount of Vitex agnus-castus, and is administrated during the follicular phase.

1. A kit for treating pelvic pain arising from the female reproductive system, the kit comprising
(a) a first pharmaceutical composition comprising a first cannabinoid and optionally a first terpene and/or a first additive;
(b) a second pharmaceutical composition comprising at least one selected from the group consisting of a second cannabinoid, a second terpene, a second additive and combinations thereof; and
(c) optionally a third pharmaceutical composition comprising a second cannabinoid and optionally a third terpene and/or a third additive, wherein a dose of said second cannabinoid in said third pharmaceutical composition is at least twice the dose of said second cannabinoid in said second pharmaceutical composition; and
(d) optionally a fourth pharmaceutical composition comprising said second cannabinoid and optionally a fourth terpene and/or a fourth additive.

2-3. (canceled)

4. The kit of claim 1, wherein a dose of said first cannabinoid in said first pharmaceutical composition is at least twice the dose of said second cannabinoid in said second pharmaceutical composition.

5-8. (canceled)

9. The kit of claim 1, wherein said second additive, when present, is different from said first additive, when present.

10. The kit of claim 1, wherein said fourth terpene, when present, is different from said third terpene, when present.

11. The kit of claim 1, wherein said fourth additive, when present, is different from said third additive, when present.

12. The kit of claim 1, wherein at least one of said first cannabinoid and said second cannabinoid, when present, is selected from the group consisting of THC, CBD, CBG, CBC, CBN, CBL, THCV and/or CBDV.

13-14. (canceled)

15. The kit of claim 1, wherein at least one of said first terpene, when present, said second terpene, when present, said third terpene, when present, and said fourth terpene, when present, is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

16. The kit of claim 1, wherein at least one of said first terpene, when present, and said third terpene, when present, is selected from the group consisting of caryophyllene, limonene, myrcene, nerolidol, linalool, terpineol, pinene, humulene, bisabolol, guaiol and combinations thereof.

17. The kit of claim 1, wherein at least one of said second terpene, when present, and said fourth terpene, when present, is selected from the group consisting of linalool, bisabolol, terpineol, myrcene, humulene and combinations thereof.

18. The kit of claim 1, wherein at least one of said first additive, when present, said second additive, when present, said third additive, when present, and said fourth additive, when present, comprises a herbal preparation selected from the group consisting of extracts of Caulophyllum thalictroides, Chamaelirium lutea, Cimicifuga (Actaea) racemosa, Dioscorea villosa spp., Paeonia lactiflora, Viburnum opulus, Viburnum prunifolium, Vitex agnus castus, Angelica sinensis, Trifolium pratense, Piscidia erythrina, Tanacetum parthenium, Chamaelirium lutea and combinations thereof.

19. The kit of claim 1, wherein at least one of said first additive, when present, said second additive, when present, said third additive, when present, and said fourth additive, when present comprises a Vitex agnus-castus and/or Chamaelirium lutea preparation.

20. The kit of claim 1, wherein at least one of said second additive, when present and said fourth additive, when present, comprises a Vitex agnus-castus and/or Chamaelirium lutea preparation.

21. The kit of claim 1, wherein at least one of said first pharmaceutical composition, said second pharmaceutical composition, said third pharmaceutical composition and said fourth pharmaceutical composition, when present, is provided in a form selected from the group consisting of a topical, a cream, a patch, a vaginal applicator, a tablet, a gel capsule, a sublingual oil, a spray, a tincture, or combinations thereof.

22. The kit of claim 1, wherein at least one of said first pharmaceutical composition and said third pharmaceutical composition is provided in a form selected from the group consisting of a topical, a cream, a patch, a tablet, a gel capsule, a sublingual oil, a spray, a tincture, or combinations thereof.

23. The kit of claim 1, for monthly use.

24. The kit of claim 1, wherein at least one of said first pharmaceutical composition and said third pharmaceutical composition comprises between 1 milligram and 100 milligrams of said cannabinoid.

25. A method for treating a subject with pelvic pain arising from female reproductive system during a menstrual cycle, comprising use of the kit of claim 1.

26. The method of claim 25, wherein said pelvic pain comprises dysmenorrhea.

27. A method for treating a subject with pelvic pain arising from female reproductive system during a menstrual cycle, comprising providing the kit of claim 1.

28. The method of claim 27, wherein said pelvic pain arises from endometriosis.

29-60. (canceled)
